(19) United States
(12) Patent Application Publication
(10) Pub. No.: US 2026/0363856 A1
(43) Pub. Date: Nov. 19, 2026

(54) DEDICATED VALVE-IN-VALVE TRANSCATHETER HEART VALVE

(71) Applicant: Sheba Impact Ltd., Ramat Gan (IL)

(72) Inventors: Israel BARBASH, Shoham (IL); Amit SEGEV, RaAnana (IL)

(73) Assignee: Sheba Impact Ltd., Ramat Gan (IL)

(21) Appl. No.: 19/121,843

(22) PCT Filed: Oct. 16, 2023
(86) PCT No.: PCT/IL2023/051080

§ 371 Date: Apr. 17, 2025

(30) Foreign Application Priority Data
Oct. 20, 2022 (IL) 297471

(51) Int. Cl. A61F 2/24

(52) U.S. Cl. CPC A61F 2/2436; A61F 2/2418; A61F 2220/0016; A61F 2220/0075; A61F 2230/0069; A61F 2250/0069

(57) ABSTRACT

The invention relates to a prosthetic heart valve having: a body having a first proximal end and a first distal end; the body having a ring located at the first distal end; one or more extending arms extending distally from the ring, each having a second proximal end and a second distal end; where only the second proximal end, from the second proximal end and the second distal end, is connected to the prosthetic heart valve; where each extending arm is a flexible arm configured to bend from a distal position to a proximal position and vice-versa; where the one or more extending arms are configured to engage leaflets and cause inward folding of the leaflets during implantation of the prosthetic heart valve; where said inward folding is caused by said bending of said flexible arms from said distal position to said proximal position.

### RELATED APPLICATION/S

This application claims the benefit of priority of Israeli Patent Application No. 297471filed on 20 Oct. 2022, the contents of which are incorporated herein by reference in their entirety.

### FIELD AND BACKGROUND OF THE INVENTION

The present invention, in some embodiments thereof, relates to a transcatheter heart valve and, more particularly, but not exclusively, to a valve-in-valve transcatheter heart valve.

It is estimated that the prevalence of valvular heart disease is currently affecting >100 million patients worldwide and with the population aging, the prevalence of this disease will further increase. More than 200,000 heart valve replacement surgeries are performed annually worldwide, with a predicted increment to 850,000 per year by 2050 about half of which involve the aortic valve. Two main types of heart valves are implanted during surgery—mechanical heart valve and bioprosthetic heart valves. The main advantage of the mechanical heart valves is their durability. Theoretically, mechanical heart valves can remain functioning throughout lifetime of the patient, however, the main limitation of these valves is the fact that they require anticoagulation treatment to prevent thrombus formation and complications. While, at younger ages the risk of bleeding during anticoagulation treatment is small—as patients become older this risk increases dramatically due to age and accumulating comorbidities and this may become a major limitation in managing patients with mechanical heart valve. Conversely, patients who receive a bioprosthetic heart valve are not required to be treated by anticoagulation and thus are not committed for lifelong anticoagulation and risk of bleeding. The main sources for bioprosthetic heart valves are bovine or porcine pericardia which are pretreated with fixatives and detergents to improve leaflet durability. The onset of structural valve deterioration of bioprosthetic heart valves generally occurs 7 to 8 years after the index implantation, with freedom from structural valve deterioration rates substantially decreasing 10 to 15 years after surgery. Considering these limitations, the past practice was to utilize mechanical heart valves in younger patients in order to provide lifelong remedy and avoid repeated heart surgeries to replace failing bioprosthetic heart valve failure. Bioprosthetic heart valves were utilized in older patients with shorter predictable lifetime in which there is reasonable probability that the bioprosthetic heart valve durability will extend beyond the patient survival.

In the last two decades, new approach to treat aortic valve stenosis has been developed, introduced into clinical practice and gained growing acceptance—this approach is based on transcatheter aortic valve replacement. At its basis—a bioprosthetic heart valve is sutured into metallic or nitinol frame, and for the purpose of valve deliverability it is crimped onto a valve delivery system and upon reaching the appropriate position at the level of the native failing aortic valve, the transcatheter aortic valve is deployed pushing away the native leaflets, and immediately beginning to function as a new aortic valve. Currently two main types of THVs. The first is a balloon expandable transcatheter heart valve called SAPIEN 3™, by Edwards Lifesciences™, as shown for example in FIG. 1a, in which the bioprosthetic heart valve in sutured into a cobalt chromium stent frame. The crimped transcatheter heart valve is loaded onto a balloon within the delivery system and the valve is deployed by inflating the balloon which expands the stent frame and the transcatheter heart valve. The second type of transcatheter heart valves is a self-expandable valve among which are included Evolut™ (Medtronic™), as shown for example in FIG. 1b, Accurate Neo™ (Boston Scientific™) as shown for example in FIG. 1c, and Portico™ (Abbott™), as shown for example in FIG. 1d. In these transcatheter heart valves the bioprosthetic valve is sutured within a nitinol frame and crimped into a delivery capsule on top of the delivery system. Upon positioning inside the aortic valve the capsule is withdrawn thus exposing the transcatheter heart valve and frame—due to the properties of nitinol—in body temperature the frame expands thus opening the transcatheter heart valve within the frame—and the valve becomes operational.

In parallel with the establishment of transcatheter valves to successfully treated native aortic valve stenosis, a trans-catheter valve in valve technique to treat degenerated surgical aortic bioprosthetic valves has gained popularity and clinical experience with excellent short and long term outcomes. The introduction of transcatheter heart valves to treat failed bioprosthetic valves, has changed the paradigm of mechanical versus bioprosthetic heart valve selection as it is now possible to treat failing bioprosthetic heart valve with transcatheter heart valve without the need for additional surgery. Thus physicians are less reluctant to utilize bioprosthetic heart valves even for younger patients. This trend shift is expected to dramatically increase the number of implanted bioprosthetic heart valves on behalf of the mechanical heart valves. Accordingly, with the increasing numbers of implanted bioprosthetic heart valves in younger patients, the numbers of failing bioprosthetic valves is expected to grow significantly in the upcoming years posing a growing clinical challenge for treating these failing valves. A trans-catheter valve in valve technique has 2 major limitations: 1) risk of coronary occlusion, and 2) residual high gradients, therefore limiting its use in 20-30% of patients.

Additional background art includes European Patent N. EP1603493B1 disclosing a prosthetic heart valve having an internal support frame with a continuous, undulating leaflet frame defined therein. The leaflet frame has three cusp regions positioned at an inflow end intermediate three commissure regions positioned at an outflow end thereof. The leaflet frame may be cloth covered and flexible leaflets attached thereto form occluding surfaces of the valve. The support frame further includes three cusp positioners rigidly fixed with respect to the leaflet frame and located at the outflow end of the support frame intermediate each pair of adjacent commissure regions. In the aortic valve position, the cusp positioners angle outward into contact with the sinus cavities, and compress the native leaflets if they are not excised, or the aortic wall if they are.

U.S. Patent Application N. US2022160502A1, disclosing a stent for interventional valve-in-valve, wherein the stent is a metal mesh tube, and is provided with four rows of transversely extending circumferential struts and a plurality of columns of axial struts arranged between the circumferential struts; the axial struts in each row are arranged in a staggered mode, the axial struts are connected with transverse struts attached thereon to form a staggered honeycomb meshes, the area of honeycomb meshes at the inflow end is basically the same as that of the honeycomb meshes in the middle row, and the area of honeycomb meshes at the outflow end is slightly larger than that of the honeycomb meshes in the other three rows. According to a stent for an interventional valve-in-valve provided therein, in view of the specialty that interventional valve-in-valves are implanted into the previously implanted damaged surgical valve or interventional valve by intervention and in close attachment with the failed valve, the subversive improvement is carried out on the conventional interventional valve stent, with all meshes of the stent adopting honeycomb-like structures, so that the stent with the structure can realize certain rigidity, has high synchronous deployment speed, good attachment, and potentially better use effect.

U.S. Patent Application N. US2022160503A1 disclosing a connecting structure of a stent and a valve leaflet which is configured such that the stent is a metal mesh tube and the valve leaflets are three fan-shaped valve leaflets arranged on the inner side of the stent. Each of the three fan-shaped valve leaflets has a free edge, an arc-shaped bottom edge and valve leaflet junction connecting parts extending on two sides. Three connecting posts are uniformly distributed on the metal mesh tube. The junction connecting part of the fan-shaped valve leaflets includes a radial overturning connecting part and an axial overturning connecting part. The radial overturning connecting part of each fan-shaped valve leaflet penetrates through the connecting post from the inner side to the outer side then folds. After the inner side of the connecting post is folded, the axial overturning connecting part is connected and fixed to the connecting post through a suture.

U.S. Pat. No. 11,357,626B2 disclosing heart valve prostheses, delivery devices, actuation handles, and other improved devices and methods that facilitate delivery of a heart valve prosthesis to a defective native valve structure in a patient, such as the aortic valve, in which native valve leaflets are sandwiched between the support frame and the valve anchor, and the likelihood of the native valve leaflets blocking the opening of the coronary artery is reduced, which may be beneficial for patients with low coronary ostia distance, and in patients with an existing valve prosthesis, who may need a new valve prosthesis inside the existing valve prosthesis (valve-in-valve application).

U.S. Patent Application N. US20170000603A1 disclosing a hybrid prosthetic heart valve configured to replace a native heart valve and having a support frame configured to be expanded post implant in order to receive and/or support an expandable prosthetic heart valve therein (a valve-in-valve procedure). The prosthetic heart valve may be configured to have a generally rigid and/or expansion-resistant configuration when initially implanted to replace a native valve (or other prosthetic heart valve), but to assume a generally expanded form when subjected to an outward force such as that provided by a dilation balloon or other mechanical expander. An inflow stent frame is expandable for anchoring the valve in place, and may have an outflow end that is collapsible for delivery and expandable post-implant to facilitate a valve-in-valve procedure.

U.S. Patent Application N. US20220183830A1 disclosing a prosthetic aortic valve intended for native or valve-in-valve within bioprostheses includes an expandable support scaffold and valve leaflets disposed within an upper leaflet portion of the support scaffold. The valve leaflets within the upper portion may be located within the annulus (intravalvular), above the annulus, or above the native or prosthetic leaflets (supravalvular). The valve within a previously implanted degenerated heart valve such that a base or lower portion of the replacement valve is within the previously implanted valve and the upper portion is expanded within the aorta, the internal area of the valve can be increased and the hemodynamics of the valve improved. Alternatively, the valve may include separate upper and lower portions allowing the portions to be implanted sequentially and the length and other characteristics of the valve to be adjusted based on patient anatomy and condition.

### SUMMARY OF THE INVENTION

Following is a non-exclusive list including some examples of embodiments of the invention. The invention also includes embodiments which include fewer than all the features in an example and embodiments using features from multiple examples, also if not expressly listed below.

Example 1. A prosthetic heart valve, comprising:
a. a body, comprising a first proximal end and a first distal end; said body comprising a ring located said first distal end;
b. one or more prosthetic leaflets attached to said body;
c. one or more extending arms, each comprising an elongated body comprising a second proximal end and a second distal end; said second proximal end connected to said ring;
wherein only said second proximal end, from said second proximal end and said second distal end, is connected to said prosthetic heart valve;
wherein each extending arm from said one or more extending arms extend distally from said ring;
wherein each extending arm is a flexible arm configured to bend from a distal position to a proximal position and vice-versa.

Example 2. The prosthetic heart valve according to example 1, wherein said second distal end of said one or more extending arms comprise a first plurality of engaging elements.

Example 3. The prosthetic heart valve according to example 1 or example 2, wherein said first plurality of engaging elements are one or more of hooks, protrusions, teeth and barbs.

Example 4. The prosthetic heart valve according to any one of examples 1-3, wherein said body comprises a second plurality of engaging elements.

Example 5. The prosthetic heart valve according to any one of examples 1-4, wherein said second plurality of engaging elements are one or more of hooks, protrusions, teeth and barbs.

Example 6. The prosthetic heart valve according to any one of examples 1-5, wherein said body comprises a length of from about 1 cm to about 20 cm.

Example 7. The prosthetic heart valve according to any one of examples 1-6, wherein said ring comprises a diameter of from about 10 mm to about 50 mm.

Example 8. The prosthetic heart valve according to any one of examples 1-7, wherein said body comprises a diameter at said proximal end of from about 10 mm to about 50 mm.

Example 9. The prosthetic heart valve according to any one of examples 1-8, wherein said body comprises a plurality of apertures.

Example 10. The prosthetic heart valve according to any one of examples 1-9, wherein said apertures comprise a height of from about 1 mm to about 50 mm and a width of from about 1 mm to about 20 mm.

Example 11. The prosthetic heart valve according to any one of examples 1-10, wherein said second plurality of engaging elements extend from the most proximal end of the device a distance of from about 0.5 cm to about 20 cm.

Example 12. The prosthetic heart valve according to any one of examples 1-11, wherein said second plurality of engaging elements extend from about a third to about two thirds of a distance as measure from the proximal end of said body.

Example 13. The prosthetic heart valve according to any one of examples 1-12, wherein said body further comprises a sealing skirt.

Example 14. The prosthetic heart valve according to any one of examples 1-13, wherein said sealing skirt extend from about a third to about two thirds of a distance as measure from the distal end of said body.

Example 15. The prosthetic heart valve according to any one of examples 1-14, wherein said sealing skirt extend from the most distal end of the device a distance of from about 1 cm to about 15 cm.

Example 16. The prosthetic heart valve according to any one of examples 1-15, wherein said one or more extending arms comprise a length of from about 0.25 cm to about 10 cm.

Example 17. The prosthetic heart valve according to any one of examples 1-16, wherein said first plurality of engaging elements extend along said one or more extending arms from an extremity of said one or more extending arms a distance of from about 0.25 mm to about 100 mm.

Example 18. The prosthetic heart valve according to any one of examples 1-17, wherein said first plurality of engaging elements do not commence at an extremity of said one or more extending arms.

Example 19. The prosthetic heart valve according to any one of examples 1-18, wherein said first plurality of engaging elements commence at a distance below an extremity of said one or more extending arms of from about 0.1 mm to about 1.5 mm.

Example 20. The prosthetic heart valve according to any one of examples 1-19, wherein said one or more extending arms are one or more extending structures extending from said ring.

Example 21. The prosthetic heart valve according to any one of examples 1-20, wherein said one or more extending structures are semi-circular structures attached to said ring.

Example 22. A method of performing a valve-in-valve procedure with a prosthetic heart valve according to example 1, the method comprising:
a. deploying said one or more extending arms from a valve delivery system towards an existing prosthetic heart valve;
b. engaging said one or more extending arms with prosthetic leaflets of said existing prosthetic heart valve;
c. advancing said ring of said prosthetic heart valve beyond a ring of said existing prosthetic heart valve while bending said one or more extending arms from a distal position to a proximal position;
d. in-folding said prosthetic leaflets of said existing prosthetic heart valve to be positioned between said prosthetic heart valve and said existing prosthetic heart valve;

e. releasing said prosthetic heart valve from said valve delivery system.

Example 23. The method according to example 22, further comprising bringing said valve delivery system close to said existing prosthetic heart valve.

Example 24. The method according to example 22 or example 23, further comprising aligning said one or more extending arms with said prosthetic leaflets of said existing prosthetic heart valve before said engaging.

Example 25. The method according to any one of examples 22-24, further comprising engaging of said second plurality of engaging elements of said prosthetic heart valve with said existing prosthetic heart valve during said releasing.

Example 26. The method according to any one of examples 22-25, wherein said engaging comprises engaging said first plurality of engaging elements with said prosthetic leaflets of said existing prosthetic heart valve.

Example 27. A prosthetic heart valve, comprising:
a. a body, comprising a first proximal end and a first distal end; said body comprising a ring located said first distal end;
b. one or more extending arms, each comprising a second proximal end and a second distal end; said second proximal end connected to said ring;
wherein said one or more extending arms are configured to engage leaflets and cause inward folding of said leaflets during implantation of said prosthetic heart valve;
wherein only said second proximal end, from said second proximal end and said second distal end, is connected to said prosthetic heart valve;
wherein each extending arm from said one or more extending arms extend distally from said ring;
wherein each extending arm is a flexible arm configured to bend from a distal position to a proximal position and vice-versa; wherein said inward folding is caused by said bending of said flexible arms from said distal position to said proximal position.

Example 28. The prosthetic heart valve according to example 27, further comprising one or more prosthetic leaflets attached to said body.

Example 29. The prosthetic heart valve according to example 27 or example 28, wherein said second distal end of said one or more extending arms comprise a first plurality of engaging elements.

Example 30. The prosthetic heart valve according to any one of examples 27-29, wherein said first plurality of engaging elements are one or more of hooks, protrusions, teeth and barbs.

Example 31. The prosthetic heart valve according to any one of examples 27-30, wherein said body comprises a second plurality of engaging elements.

Example 32. The prosthetic heart valve according to any one of examples 27-31, wherein said second plurality of engaging elements are one or more of hooks, protrusions, teeth and barbs. Example 33. The prosthetic heart valve according to any one of examples 27-32, wherein said body comprises a length of from about 1 cm to about 20 cm.

Example 34. The prosthetic heart valve according to any one of examples 27-33, wherein said ring comprises a diameter of from about 10 mm to about 50 mm.

Example 35. The prosthetic heart valve according to any one of examples 27-34, wherein said body comprises a diameter at said proximal end of from about 10 mm to about 50 mm.

Example 36. The prosthetic heart valve according to any one of examples 27-35, wherein said body comprises a plurality of apertures.

Example 37. The prosthetic heart valve according to any one of examples 27-36, wherein said apertures comprise a height of from about 1 mm to about 50 mm and a width of from about 1 mm to about 20 mm.

Example 38. The prosthetic heart valve according to any one of examples 27-37, wherein said second plurality of engaging elements extend from the most proximal end of the device a distance of from about 0.5 cm to about 20 cm.

Example 39. The prosthetic heart valve according to any one of examples 27-38, wherein said second plurality of engaging elements extend from about a third to about two thirds of a distance as measure from the proximal end of said body.

Example 40. The prosthetic heart valve according to any one of examples 27-39, wherein said body further comprises a sealing skirt;
wherein said sealing skirt extend from about a third to about two thirds of a distance as measure from the distal end of said body; and
wherein said sealing skirt extend from the most distal end of the device a distance of from about 1 cm to about 15 cm.

Example 41. The prosthetic heart valve according to any one of examples 27-40, wherein said one or more extending arms comprise a length of from about 0.25 cm to about 10 cm.

Example 42. The prosthetic heart valve according to any one of examples 27-41, wherein said first plurality of engaging elements extend along said one or more extending arms from an extremity of said one or more extending arms a distance of from about 0.25 mm to about 100 mm.

Example 43. The prosthetic heart valve according to any one of examples 27-42, wherein said first plurality of engaging elements do not commence at an extremity of said one or more extending arms.

Example 44. The prosthetic heart valve according to any one of examples 27-43, wherein said first plurality of engaging elements commence at a distance below an extremity of said one or more extending arms of from about 0.1 mm to about 1.5 mm.

Example 45. The prosthetic heart valve according to any one of examples 27-44, wherein said one or more extending arms are one or more extending structures extending from said ring.

Example 46. The prosthetic heart valve according to any one of examples 27-45, wherein said one or more extending structures are semi-circular structures attached to said ring.

Example 47. An add-on device for a prosthetic heart valve, comprising:
a. a ring;
b. one or more extending arms, each comprising a proximal end and a distal end; said proximal end connected to said ring;
wherein said one or more extending arms are configured to engage leaflets and cause inward folding of said leaflets during implantation of said add-on device;
wherein only said proximal end, from said proximal end and said distal end, is connected to said prosthetic heart valve;
wherein each extending arm from said one or more extending arms extend distally from said ring;
wherein each extending arm is a flexible arm configured to bend from a distal position to a proximal position and vice-versa wherein said inward folding is caused by said bending of said flexible arms from said distal position to said proximal position.

Example 48. The add-on device according to example 47, further comprising a prosthetic heart valve comprising a body and one or more prosthetic leaflets attached to said body, wherein said add-on is configured to be coupled to said prosthetic heart valve.

Example 1001. A prosthetic heart valve, comprising:
a. a body, comprising a first proximal end and a first distal end; said body comprising a ring located said first distal end;
b. one or more prosthetic leaflets attached to said body;
c. one or more extending arms, each comprising a second proximal end and a second distal end; said second proximal end connected to said ring.
Example 1002. The prosthetic heart valve according to example 1001, wherein said second distal end of said one or more extending arms comprise a first plurality of engaging elements.
Example 1003. The prosthetic heart valve according to example 1002, wherein said first plurality of engaging elements are one or more of hooks, protrusions, teeth and barbs.
Example 1004. The prosthetic heart valve according to any one of examples 1001-1003, wherein said one or more extending arms are flexible arms configured to bend from a distal position to a proximal position and vice-versa.
Example 1005. The prosthetic heart valve according to any one of examples 1001-1004, wherein said body comprises a second plurality of engaging elements.
Example 1006. The prosthetic heart valve according to example 1005, wherein said second plurality of engaging elements are one or more of hooks, protrusions, teeth and barbs.
Example 1007. The prosthetic heart valve according to any one of examples 1001-1006, wherein said body comprises a length of from about 1 cm to about 20 cm.

Example 1008. The prosthetic heart valve according to any one of examples 1001-1007, wherein said ring comprises a diameter of from about 10 mm to about 50 mm.

Example 1009. The prosthetic heart valve according to any one of examples 1001-1008, wherein said body comprises a diameter at said proximal end of from about 10 mm to about 50 mm.

Example 1010. The prosthetic heart valve according to any one of examples 1001-1009, wherein said body comprises a plurality of apertures.

Example 1011. The prosthetic heart valve according to example 1010, wherein said apertures comprise a height of from about 1 mm to about 50 mm and a width of from about 1 mm to about 20 mm.

Example 1012. The prosthetic heart valve according to example 1005, wherein said second plurality of engaging elements extend from the most proximal end of the device a distance of from about 0.5 cm to about 20 cm.

Example 1013. The prosthetic heart valve according to example 1005, wherein said second plurality of engaging elements extend from about a third to about two thirds of a distance as measure from the proximal end of said body.

Example 1014. The prosthetic heart valve according to any one of examples 1001-1013, wherein said body further comprises a sealing skirt.

Example 1015. The prosthetic heart valve according to example 1014, wherein said sealing skirt extend from about a third to about two thirds of a distance as measure from the distal end of said body.

Example 1016. The prosthetic heart valve according to example 1014, wherein said sealing skirt extend from the most distal end of the device a distance of from about 1 cm to about 15 cm.

Example 1017. The prosthetic heart valve according to any one of examples 1001-1016, wherein said one or more extending arms comprise a length of from about 0.25 cm to about 10 cm.

Example 1018. The prosthetic heart valve according to any one of examples 1001-1017, wherein said first plurality of engaging elements extend along said one or more extending arms from an extremity of said one or more extending arms a distance of from about 0.25 mm to about 100 mm.

Example 1019. The prosthetic heart valve according to any one of examples 1001-1018, wherein said first plurality of engaging elements do not commence at an extremity of said one or more extending arms.

Example 1020. The prosthetic heart valve according to any one of examples 1001-1019, wherein said first plurality of engaging elements commence at a distance below an extremity of said one or more extending arms of from about 0.1 mm to about 1.5 mm.

Example 1021. The prosthetic heart valve according to any one of examples 1001-1020, wherein said one or more extending arms are one or more extending structures extending from said ring.

Example 1022. The prosthetic heart valve according to example 1021, wherein said one or more extending structures are semi-circular structures attached to said ring.

Example 1023. A method of performing a valve-in-valve procedure with a prosthetic heart valve according to example 1, the method comprising:
a. deploying said one or more extending arms from a valve delivery system towards an existing prosthetic heart valve;
b. engaging said one or more extending arms with prosthetic leaflets of said existing prosthetic heart valve;
c. advancing said ring of said prosthetic heart valve beyond a ring of said existing prosthetic heart valve;
d. in-folding said prosthetic leaflets of said existing prosthetic heart valve to be positioned between said prosthetic heart valve and said existing prosthetic heart valve;
e. releasing said prosthetic heart valve from said valve delivery system.

Example 1024. The method according to example 1024, further comprising bringing said valve delivery system close to said existing prosthetic heart valve.

Example 1025. The method according to example 1024, further comprising aligning said one or more extending arms with said prosthetic leaflets of said existing prosthetic heart valve before said engaging.

Example 1026. The method according to example 1024, further comprising engaging of said second plurality of engaging elements of said prosthetic heart valve with said existing prosthetic heart valve during said releasing.

Example 1027. The method according to examples 1024, wherein said engaging comprises engaging said first plurality of engaging elements with said prosthetic leaflets of said existing prosthetic heart valve.

Example 1028. A prosthetic heart valve, comprising:
a. a body, comprising a first proximal end and a first distal end; said body comprising a ring located said first distal end;
b. one or more extending arms, each comprising a second proximal end and a second distal end; said second proximal end connected to said ring;

wherein said one or more extending arms are configured to engage leaflets and cause inward folding of said leaflets during implantation of said add-on device.

Example 1029. The prosthetic heart valve according to example 1028, further comprising one or more prosthetic leaflets attached to said body.

Example 1030. The prosthetic heart valve according to example 1028, wherein said second distal end of said one or more extending arms comprise a first plurality of engaging elements.

Example 1031. The prosthetic heart valve according to example 1030, wherein said first plurality of engaging elements are one or more of hooks, protrusions, teeth and barbs.

Example 1032. The prosthetic heart valve according to any one of examples 1028-1031, wherein said one or more extending arms are flexible arms configured to bend from a distal position to a proximal position and vice-versa.

Example 1033. The prosthetic heart valve according to any one of examples 1028-1032, wherein said body comprises a second plurality of engaging elements.

Example 1034. The prosthetic heart valve according to example 1033, wherein said second plurality of engaging elements are one or more of hooks, protrusions, teeth and barbs.

Example 1035. The prosthetic heart valve according to any one of examples 1028-1034, wherein said body comprises a length of from about 1 cm to about 20 cm.

Example 1036. The prosthetic heart valve according to any one of examples 1028-1035, wherein said ring comprises a diameter of from about 10 mm to about 50 mm.

Example 1037. The prosthetic heart valve according to any one of examples 1028-1036, wherein said body comprises a diameter at said proximal end of from about 10 mm to about 50 mm.

Example 1038. The prosthetic heart valve according to any one of examples 1028-1037, wherein said body comprises a plurality of apertures.

Example 1039. The prosthetic heart valve according to example 1038, wherein said apertures comprise a height of from about 1 mm to about 50 mm and a width of from about 1 mm to about 20 mm.

Example 1040. The prosthetic heart valve according to example 1033, wherein said second plurality of engaging elements extend from the most proximal end of the device a distance of from about 0.5 cm to about 20 cm.

Example 1041. The prosthetic heart valve according to example 1033, wherein said second plurality of engaging elements extend from about a third to about two thirds of a distance as measure from the proximal end of said body.

Example 1042. The prosthetic heart valve according to any one of examples 1028-1041, wherein said body further comprises a sealing skirt.

Example 1043. The prosthetic heart valve according to example 1042, wherein said sealing skirt extend from about a third to about two thirds of a distance as measure from the distal end of said body.

Example 1044. The prosthetic heart valve according to example 1042, wherein said sealing skirt extend from the most distal end of the device a distance of from about 1 cm to about 15 cm.

Example 1045. The prosthetic heart valve according to any one of examples 1028-1044, wherein said one or more extending arms comprise a length of from about 0.25 cm to about 10 cm.

Example 1046. The prosthetic heart valve according to any one of examples 2108-1045, wherein said first plurality of engaging elements extend along said one or more extending arms from an extremity of said one or more extending arms a distance of from about 0.25 mm to about 100 mm.

Example 1047. The prosthetic heart valve according to any one of examples 1028-1046, wherein said first plurality of engaging elements do not commence at an extremity of said one or more extending arms.

Example 1048. The prosthetic heart valve according to any one of examples 1028-1047, wherein said first plurality of engaging elements commence at a distance below an extremity of said one or more extending arms of from about 0.1 mm to about 1.5 mm.

Example 1049. The prosthetic heart valve according to any one of examples 1028-1048, wherein said one or more extending arms are one or more extending structures extending from said ring.

Example 1050. The prosthetic heart valve according to example 1049, wherein said one or more extending structures are semi-circular structures attached to said ring.

Example 1051. An add-on device for a prosthetic heart valve, comprising:
a. a ring;
b. one or more extending arms, each comprising a proximal end and a distal end; said proximal end connected to said ring;
wherein said one or more extending arms are configured to engage leaflets and cause inward folding of said leaflets during implantation of said add-on device.

Example 1052. The add-on device according to example 1051, wherein said distal end of said one or more extending arms comprise a first plurality of engaging elements.

Example 1053. The add-on device according to example 1052, wherein said first plurality of engaging elements are one or more of hooks, protrusions, teeth and barbs.

Example 1054. The add-on device according to any one of examples 1051-1053, wherein said one or more extending arms are flexible arms configured to bend from a distal position to a proximal position and vice-versa.

Example 1055. The add-on device according to any one of examples 1051-1054, wherein said ring comprises a diameter of from about 10 mm to about 50 mm.

Example 1056. The add-on device according to any one of examples 1051-1055, wherein said one or more extending arms comprise a length of from about 0.25 cm to about 10 cm.

Example 1057. The add-on device according to any one of examples 1051-1056, wherein said first plurality of engaging elements extend along said one or more extending arms from an extremity of said one or more extending arms a distance of from about 0.25 mm to about 100 mm.

Example 1058. The add-on device according to any one of examples 1051-1057, wherein said first plurality of engaging elements do not commence at an extremity of said one or more extending arms.

Example 1059. The add-on device according to any one of examples 1051-1058, wherein said first plurality of engaging elements commence at a distance below an extremity of said one or more extending arms of from about 0.1 mm to about 1.5 mm.

Example 1060. The add-on device according to any one of examples 1051-1059, wherein said one or more extending arms are one or more extending structures extending from said ring.

Example 1061. The add-on device according to example 1060, wherein said one or more extending structures are semi-circular structures attached to said ring.

Example 1062. The add-on device according to any one of examples 1051-1061, further comprising a prosthetic heart valve comprising a body and one or more prosthetic leaflets attached to said body, wherein said add-on is configured to be coupled to said prosthetic heart valve.

Example 1063. The add-on device according to example 1062, wherein said body comprises a second plurality of engaging elements.

Example 1064. The add-on device according to example 1063, wherein said second plurality of engaging elements are one or more of hooks, protrusions, teeth and barbs.

Example 1065. The add-on device according to any one of examples 1062-1064, wherein said body comprises a length of from about 1 cm to about 20 cm.

Example 1066. The add-on device according to any one of examples 1062-1065, wherein said body comprises a diameter at said proximal end of from about 10 mm to about 50 mm.

Example 1067. The add-on device according to any one of examples 1062-1066, wherein said body comprises a plurality of apertures.

Example 1068. The add-on device according to example 1067, wherein said apertures comprise a height of from about 1 mm to about 50 mm and a width of from about 1 mm to about 20 mm.

Example 1069. The add-on device according to example 1063, wherein said second plurality of engaging elements extend from the most proximal end of the device a distance of from about 0.5 cm to about 20 cm.

Example 1070. The add-on device according to example 63, wherein said second plurality of engaging elements extend from about a third to about two thirds of a distance as measure from the proximal end of said body.

Example 1071. The add-on device according to any one of examples 1062-1070, wherein said body further comprises a sealing skirt.

Example 1072. The add-on device according to example 1071, wherein said sealing skirt extend from about a third to about two thirds of a distance as measure from the distal end of said body.

Example 1073. The add-on device according to example 1071, wherein said sealing skirt extend from the most distal end of the device a distance of from about 1 cm to about 15 cm.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1a-d are images of known transcatheter heart valves;

FIG. 2 is a schematic representation of an exemplary bioprosthetic heart valve, according to some embodiments of the invention;

FIGS. 3a-b are schematic representations of exemplary general mechanisms of deployment of an exemplary prosthetic heart valve, according to some embodiments of the invention;

FIGS. 4a-g are schematic representations of an exemplary method of performing a valve-in-valve implantation procedure, according to some embodiments of the invention;

FIG. 5 is a flowchart of an exemplary method of performing a valve-in-valve implantation procedure, according to some embodiments of the invention;

FIG. 6 is a schematic representation of an exemplary alternative extending structure of an exemplary bioprosthetic heart valve, according to some embodiments of the invention; and

FIGS. 7a-c are schematic representations of exemplary add-on in-folding leaflets device, according to some embodiments of the invention.

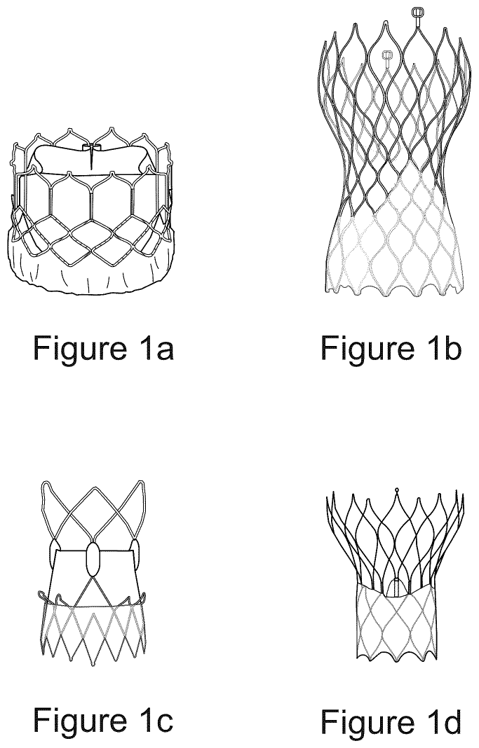

### DESCRIPTION OF SPECIFIC EMBODIMENTS OF THE INVENTION

The present invention, in some embodiments thereof, relates to a transcatheter heart valve and, more particularly, but not exclusively, to a valve-in-valve transcatheter heart valve.

### Overview

An aspect of some embodiments of the invention relates to replacement of a failing bioprosthetic heart valve by a transcatheter heart valve (also called “valve-in-valve” procedure). In some embodiments, the new transcatheter heart valve is implanted inside the old, failing bioprosthetic valve frame and not in the native annulus of the aortic valve. In some embodiments, implantation of the new heart valve does not cause coronary artery occlusion, for example due to the proximity of the failing bioprosthetic heart valve to the coronary artery ostia, especially the left main coronary artery but also the right coronary artery. In some embodiments, the transcatheter aortic valve is implanted underneath the ring (infra-annular) of the failing bioprosthetic heart valve, while providing a low residual post procedural transvalvular gradients, thereby potentially avoiding problems related to high residual gradients that may cause residual symptoms as the aortic valve stenosis was not completely resolved. In some embodiments, replacement of a failing bioprosthetic heart valve by a transcatheter heart valve comprises pushing down the old bioprosthetic leaflets. In some embodiments, pushing down means in-folding the leaflets. In known transcatheter heart valves, both balloon expandable and self expandable valves utilize the same principal that is used in a native valve replacement—the valve leaflets are pushed aside by the transcatheter heart valve frame. While this feature might be effective for native valves—this feature has significant limitations in term of short and long term coronary access. Potential advantages of pushing down (in-folding) the old bioprosthetic leaflets towards the left ventricular outflow tract are: (a) it potentially lowers the risk of coronary artery occlusion by removing the leaflets away from the coronary ostia; and (b) the downward flipped leaflets will help achieve better sealing between the failing bioprosthetic heart valve and the new transcatheter heart valve.

In some embodiments, the device used for the transcatheter aortic valve which is implanted within the ring of the failing bioprosthetic heart valve is a sub-annular valve. One of the major challenges of valve-in-valve procedures is the fact that the new implanted transcatheter heart valve is constrained within the ring of the failing bioprosthetic valve, which may lead to residual post procedure gradients across the implanted transcatheter heart valve limiting symptom improvement and transcatheter heart valve durability. Known transcatheter heart valve systems in valve-in-valve procedures comprise an intra-annular or supra-annular design. However, this type of design suffers from significant limitations in valve-in-valve procedures as the supra-annular design of the transcatheter heart valve risks coronary artery occlusion and future access to the coronary arteries. In some embodiments, a potential advantage of having a sub-annular valve is that it potentially overcomes the abovementioned limitations by allowing to provide a larger effective orifice area of the new transcatheter heart valve because it is deployed below the old bioprosthetic valve ring, while potentially not risking coronary artery occlusion and access as the new valve apparatus is located below the old bioprosthetic valve ring.

In some embodiments, the device is an add-on device comprising a ring and one or more extending arms, which is used with an existing prosthetic heart valve.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

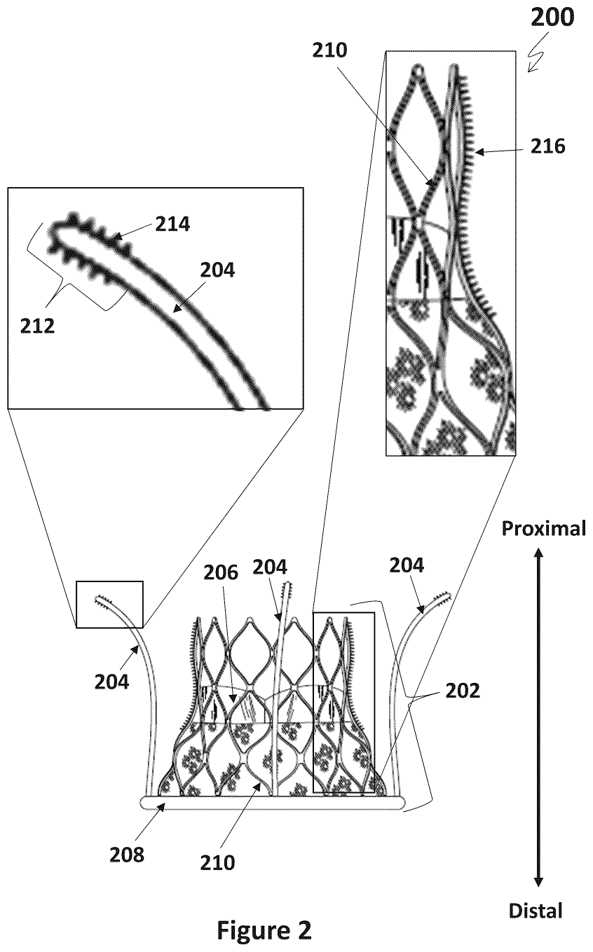

Referring now to FIG. 2, showing a schematic representation of an exemplary bioprosthetic heart valve, according to some embodiments of the invention. In the following description the terms “prosthetic heart valve”, “heart valve implant”, “valve-in-valve implant”, “device”, etc., all refer to the exemplary bioprosthetic heart valve as shown in FIG. 2, unless otherwise disclosed.

In some embodiments, the prosthetic heart valve 200 comprises a body 202, a plurality of extending arms 204 and one or more leaflets 206.

### Exemplary Body 202

In some embodiments, an exemplary body 202 of an exemplary prosthetic heart valve 200 is characterized by being a metallic frame (for example nitinol or other). In some embodiments, the metallic frame comprises a woven, knitted or braided cylindrical mesh structure made of, for example, stainless steel, nitinol and/or chrome-cobalt alloy. In some embodiments, the exemplary body 202 comprises a distal end (see arrow showing directionality) comprising a reversibly-compressible ring base 208, from which a braided cylindrical mesh structure 210 extends proximally. In some embodiments, optionally, parts of the metallic frame comprise a plurality of engaging elements 216, for example hooks, protrusions, teeth and/or barbs, configured to assist in anchoring the device to the location where is being deployed, for example within an old prosthetic heart valve.

In some embodiments, an exemplary length of the body, as measured from the most proximal side to the most distal side of the body is from about 3 cm to about 15 cm; optionally from about 2 cm to about 17 cm; optionally from about 1 cm to about 20 cm. In some embodiments, an exemplary diameter of the ring base located at the distal end of the device is from about 18 mm to about 35 mm; optionally from about 15 mm to about 40 mm; optionally from about 10 mm to about 50 mm. In some embodiments, an exemplary diameter of the device at the proximal end of the device is from about 18 mm to about 35 mm; optionally from about 15 mm to about 40 mm; optionally from about 10 mm to about 50 mm. In some embodiments, the body comprises a plurality of holes or apertures having exemplary sizes of: a height from about 5 mm to about 15 mm; optionally from about 3 mm to about 20 mm; optionally from about 1 mm to about 50 mm; and a width from about 3 mm to about 7 mm; optionally from about 2 mm to about 10 mm; optionally from about 1 mm to about 20 mm. In some embodiments, the plurality of engaging elements 216 extend from the most proximal end of the device a distance of from about 2 cm to about 10 cm; optionally from about 1 cm to about 15 cm; optionally form about 0.5 cm to about 20 cm. In some embodiments, the plurality of engaging elements 216 extend about a third of the distance measured from the proximal side of the body; optionally extend about half of the distance measured from the proximal side of the body; optionally extend about two thirds of the distance measured from the proximal side of the body; optionally from about a third to about two thirds of the distance measured from the proximal side of the body.

In some embodiments, optionally, the body comprises a sealing layer (sealing skirt) located on the outside of the body, and extends a about a third of the distance measured from the distal side of the body (for example the ring); optionally extend about half of the distance measured from the distal side of the body; optionally extend about two thirds of the distance measured from the distal side of the body optionally from about a third to about two thirds of the distance measured from the distal side of the body. In some embodiments, the sealing layer extends from the most distal side a distance of from about 2 cm to about 8 cm; optionally from about 1.5 cm to about 10 cm; optionally from about 1 cm to about 15 cm.

### Exemplary Extending Arms 204

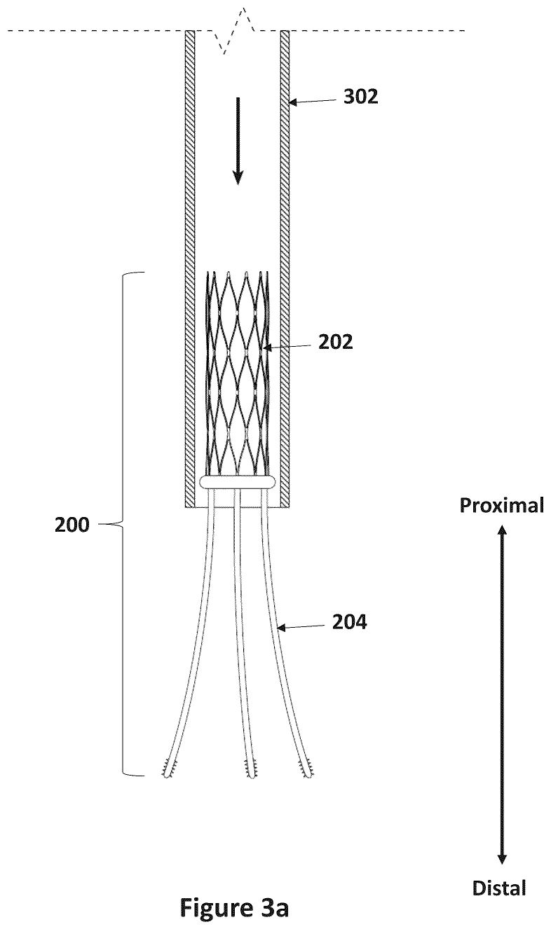

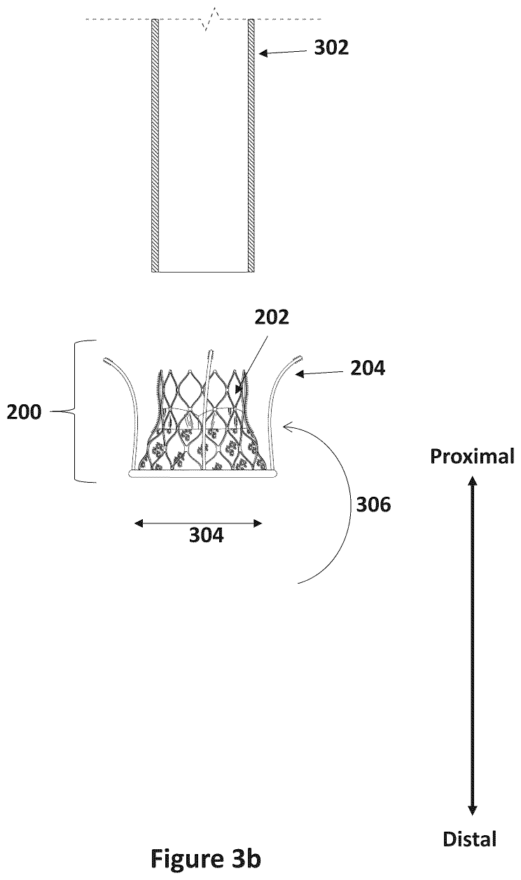

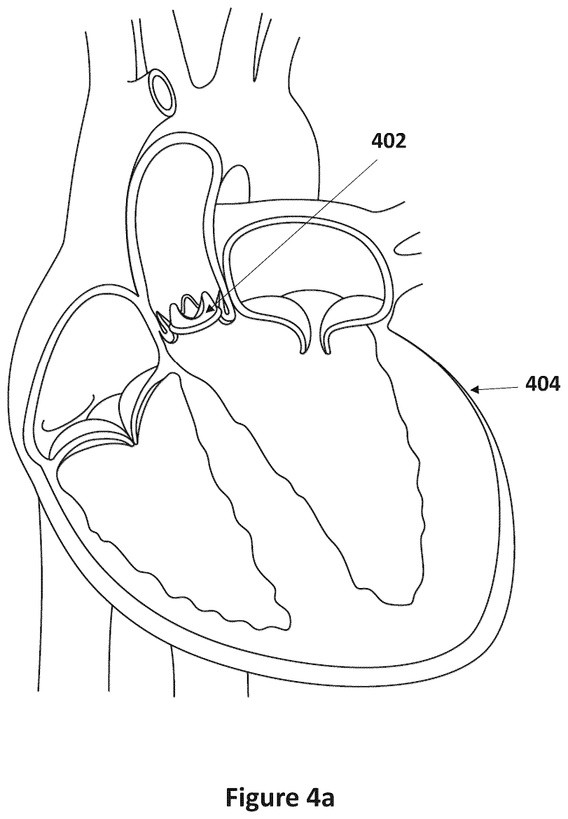

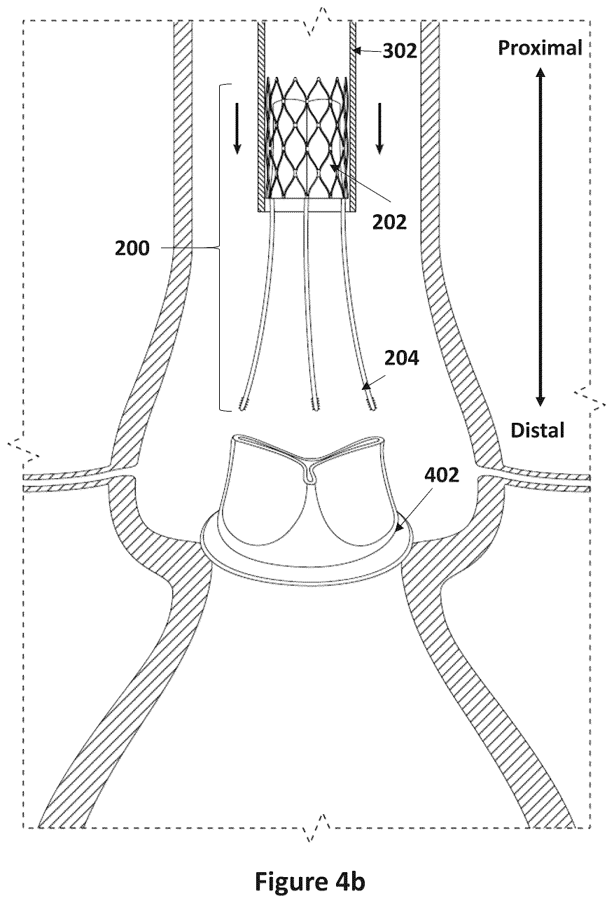

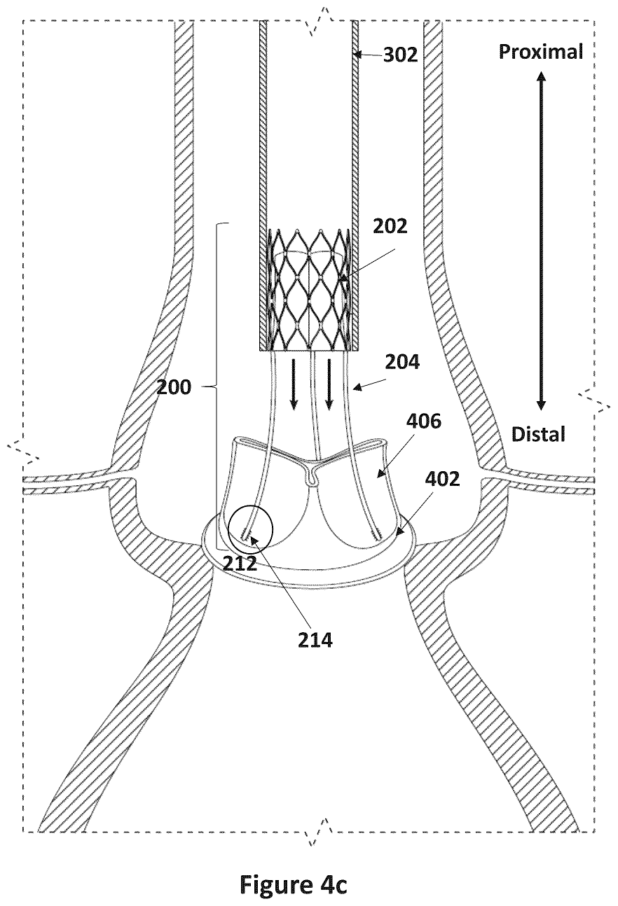

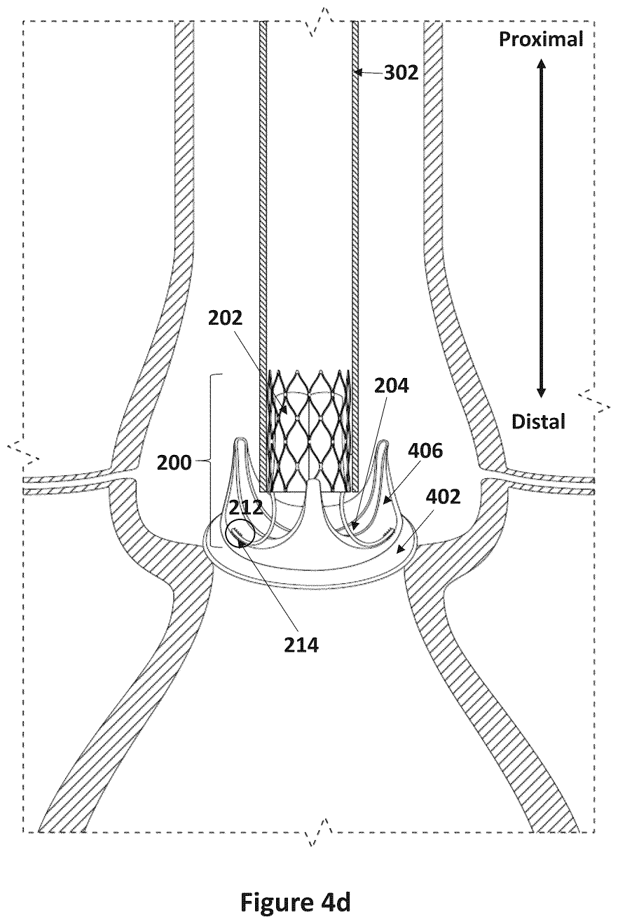

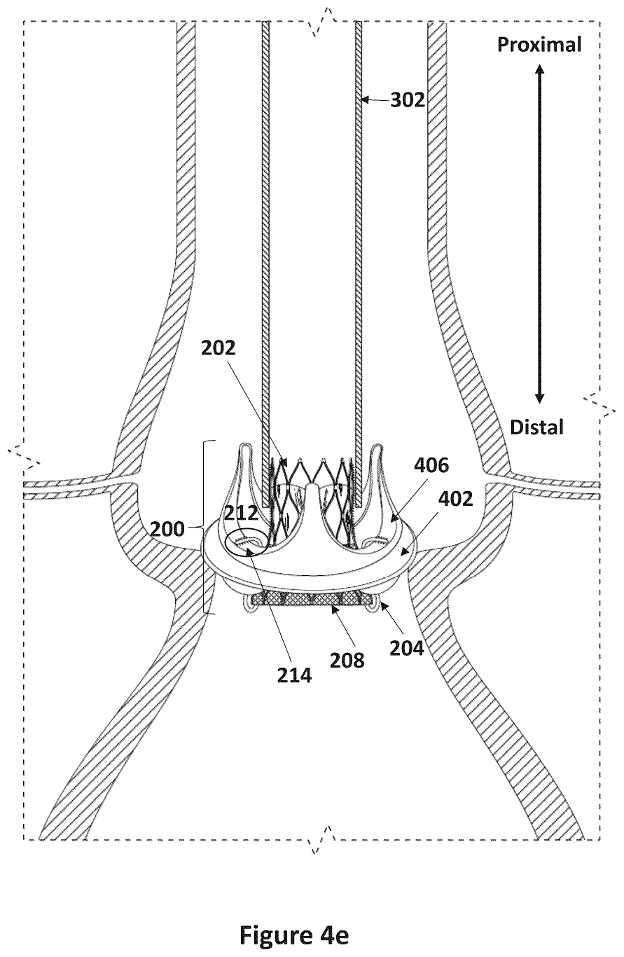

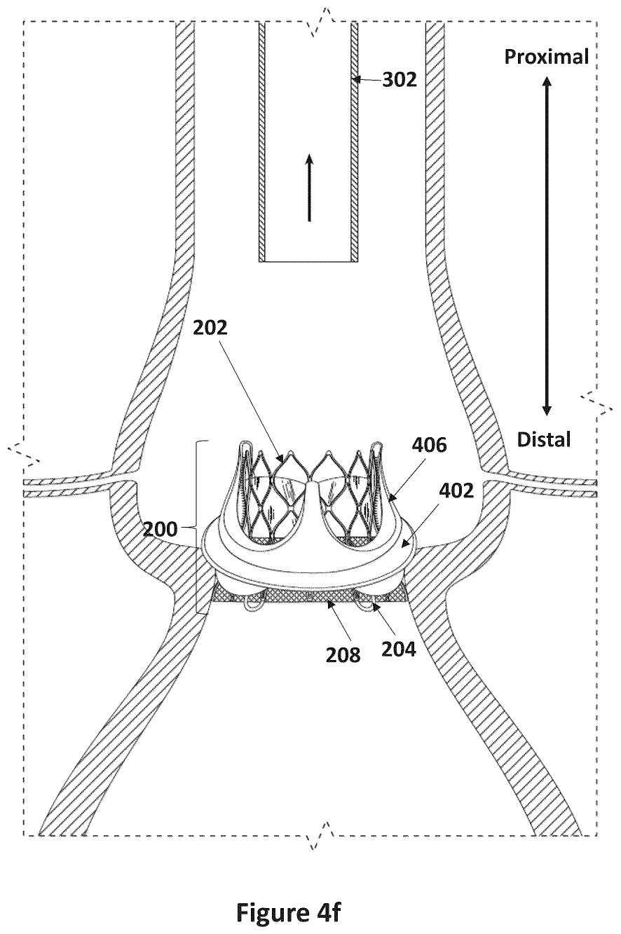

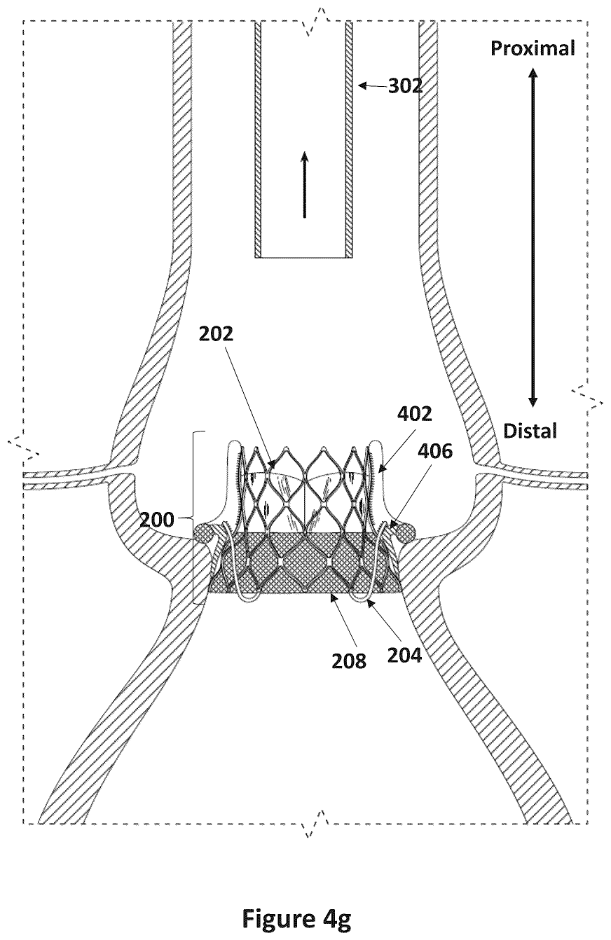

In some embodiments, the plurality of extending arms 204 are connected to the body 202 at the reversibly-compressible ring base 208. In some embodiments, one or more of the extending arms 204 are flexible arms configured to be allowed to move from a distal position to a proximal position, and vice versa. For example, in FIG. 2, the extending arms 204 are shown in a proximal position (the arms extend from the reversibly-compressible ring base 208 in a proximal direction in relation to the orientational positioning of the parts of the device as defined herein). For example, in FIG. 3a, the extending arms 204 are shown in a distal position (the arms extend from the reversibly-compressible ring base 208 in a distal direction in relation to the orientational positioning of the parts of the device as defined herein). In some embodiments, the flexibility of the extending arms 204 not only allows the arms to pivot/move/flex at the connection between the extending arms 204 and the reversibly-compressible ring base 208, but also along the body of the extending arms 204, as shown for example in FIG. 4d.

In some embodiments, at the extremity 212 of the extending arms 204, opposite to the location of the connection between the extending arms 204 and the reversibly-compressible ring base 208, the extending arms 204 are provided with a plurality of engaging elements 214, for example, hooks, protrusions, teeth and/or barbs. In some embodiments, the plurality of engaging elements extend from the extremity of the arm until at least half a distance as measured from the extremity of the extending arm 204 to the location of the connection between the extending arms 204 and the reversibly-compressible ring base 208.

In some embodiments, the plurality of engaging elements 214 comprise a length of from about 1 mm to about 2 mm. Optionally, from about 0.5 mm to about 2.5 mm. Optionally from about 0.1 mm to about 3 mm. In some embodiments, the plurality of engaging elements 214 are configured to facilitate attachment of the extending arm(s) 204 to the base of the leaflet of the old pre-existing valve (see below).

In some embodiments, the extending arms 204 comprise a length of from about 1 cm to about 4 cm; optionally from about 0.5 cm to about 6 cm; optionally from about 0.25 cm to about 10 cm. In some embodiments, the plurality of engaging elements 214 extend a distance of from about 1 mm to about 10 mm along the extending arms when measured from the extremity; optionally from about 0.5 mm to about 50 mm; optionally from about 0.25 mm to about 100 mm. In some embodiments, the plurality of engaging elements do not commence at the extremity, but below the extremity, at a distance of from about 0.5 mm to about 0.7 from the extremity; optionally from about 0.3 mm to about 1 mm; optionally from about 0.1 mm to about 1.5 mm.

In some embodiments, a potential advantage of the extending arms is that it potentially allows the in-folding of the leaflets, instead of just pushing them aside, which is beneficial because:
(a) it potentially lowers the risk of coronary artery occlusion by removing the leaflets away from the coronary ostia; and (b) the downward flipped leaflets will help achieve better sealing between the failing bioprosthetic heart valve and the new transcatheter heart valve.

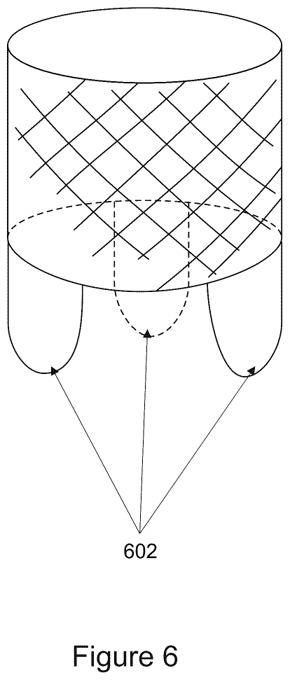

In some embodiments, instead of having extending arms, the device comprises different extending structures configured to perform the same actions, for example, instead of extending arms, the device comprises one or more semi-circular structures, 602 as shown for example in FIG. 6. In some embodiments, the semi-circular structures are made of metal, like nitinol, or made of any other material. In some embodiments, for example, there are three semi-circular structures, each sitting at the location of each of the three aortic valve sinuses. In some embodiments, the actions performed by the one or more semi-circular structures are the same as above. In some embodiments, the one or more semi-circular structures also comprise the optional engaging elements 214, for example, hooks, protrusions, teeth and/or barbs.

### Exemplary Leaflets 206

In some embodiments, the leaflets are membranes made of animal-based materials, artificial materials or a combination thereof. In some embodiments, the leaflets are connected to body at a plurality of locations, for example at the ring base of the device, at several locations over the body of the device and/or at a distal locations of the body of the device.

### Exemplary Methods

In some embodiments, as mentioned above, when an old prosthetic heart valve is in need to be replaced because, for example, it does not function anymore, there are in general two possible actions that can be performed: 1. Removing the old prosthetic heart valve and install a new one; 2. Install a new prosthetic heart valve on top (or within) the old prosthetic heart valve-a procedure called valve-in-valve placement. In many cases it is not advisable and/or possible to remove an old prosthetic heart valve, leaving only the option of performing a valve-in-valve procedure.

When performing a valve-in-valve procedure, it is advisable to take a few things under consideration. For example, it is important to “neutralize” the old prosthetic leaflets of the old prosthetic heart valve to avoid interference in the functioning of the new prosthetic leaflets of the new prosthetic heart valve. Additionally, for example, in view of the location of the implantation of the old prosthetic heart valve, the implantation of new prosthetic heart valve in a valve-in-valve procedure should not block and/or interfere with the coronary blood flow. Lastly, for example, the implantation of a new prosthetic heart valve within an old prosthetic heart valve, should not cause an excessive narrowing of the valvular channel.

In some embodiments, potential advantages of the device are one or more of the following:
1. A potential advantage of the device in certain embodiments is that it potentially effectively “neutralizes” the old prosthetic leaflets of the old prosthetic heart valve by the use of the extending arms.
2. A potential advantage of the device in certain embodiments is that it potentially allows to perform a valve-in-valve procedure without obstructing access to the coronary arteries.
3. A potential advantage of the device in certain embodiments is that it potentially allows to perform a valve-in-valve procedure without causing an excessive narrowing of the valvular channel by avoiding an excessive transvalvular gradients.

In the following paragraphs, an exemplary method of performing a valve-in-valve implantation of the exemplary device as disclosed above will be disclosed and the abovementioned potential advantages will become apparent as the process is explained.

Referring now to FIGS. 3a-3b showing an exemplary general mechanism of deployment of an exemplary prosthetic heart valve, according to some embodiments of the invention. In some embodiments, the general mechanism of deployment of an exemplary prosthetic heart valve as shown in FIG. 2 (same reference numerals are used for previously mentioned parts), is as follows:

Referring now to FIG. 3a, the body 202 of the prosthetic heart valve 200 is crimped using methods known in art, and inserted within a delivery device, for example a valve delivery system 302, while leaving the extending arms 204 in a distal position (the arms extend from the reversibly-compressible ring base 208 in a distal direction in relation to the orientational positioning of the parts of the device as defined herein).

In some embodiments, when the prosthetic heart valve 200 is pushed distally from the valve delivery system 302 in order to make it exit a distal end of the valve delivery system 302, the extending arms 204 are the first parts to be deployed. In some embodiments, as can be seen in FIG. 3a, the extending arms 204 are deployed outside the valve delivery system 302 while the body 202 of the prosthetic heart valve 200 is kept within the valve delivery system 302. In some embodiments, this allows maneuvering the extending arms 204 without the need to completely deploy the prosthetic heart valve 200. In some embodiments, this is important because of the role of the extending arms 204 in “neutralizing” the old prosthetic leaflets, as will be further explained below.

Referring now to FIG. 3b, once the body 202 of the prosthetic heart valve 200 is pushed beyond the distal end of the valve delivery system 302, the body 202 is physically allowed to expand (un-crimp), in a dimension as schematically shown by the arrow 304. In some embodiments, additionally, the extending arms 204 move to a proximal position (the arms extend from the reversibly-compressible ring base 208 in a proximal direction in relation to the orientational positioning of the parts of the device as defined herein), in a dimension as schematically shown by arrow 306. In some embodiments, the mechanism that allow and/or perform the movement of the extending arms 204 is the attachment/engagement of the extending arms 204 on the old prosthetic leaflets, as will be further described below. This means that, in some embodiments, the movement of the extending arms 204 from a distal position to a proximal position is a passive movement that occurs due to the distal movement of the prosthetic heart valve 200 when exiting the valve delivery system 302 and the fact that the engaging elements 214 located at the extremities 212 of the extending arms 204 are engaged/attached to the old prosthetic leaflets, and the extending arms 204 are configured to be flexible and allow the passive movement when forces are applied (forces due to the distal movement of the prosthetic heart valve 200).

Following the general exemplary general mechanism of deployment of the exemplary prosthetic heart valve disclosed above, an exemplary valve-in-valve procedure will be explained.

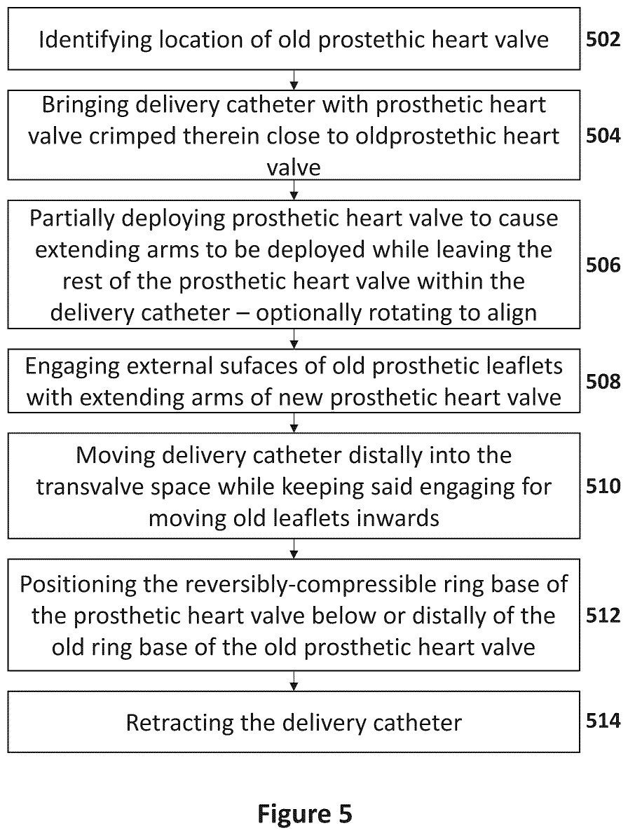

Referring now to FIGS. 4a-g, showing an exemplary method of performing a valve-in-valve implantation procedure, according to some embodiments of the invention, and to FIG. 5, showing a flowchart of the exemplary method of performing a valve-in-valve implantation procedure, according to some embodiments of the invention. Same reference numerals are used for previously mentioned parts.

In some embodiments, the location of an old prosthetic heart valve 402 is identified in the heart 404 of the patient (FIGS. 4a and 502 in FIG. 5).

In some embodiments, a valve delivery system 302 is brought to the location (or close to the location) of the old prosthetic heart valve (504 in FIG. 5).

In some embodiments, the prosthetic heart valve 200 is pushed distally to cause a partial deployment of the prosthetic heart valve 200, which comprises the deployment of the extending arms 204 from the distal end of the valve delivery system 302, as shown for example in FIG. 4b (506 in FIG. 5). In some embodiments, deployment of the extending arms 204 from the distal end of the valve delivery system 302 comprises deployment of the extending arms 204 while being in a distal position (the arms extend from the reversibly-compressible ring base 208 in a distal direction in relation to the orientational positioning of the parts of the device as defined herein).

In some embodiments, optionally, the valve delivery system is rotated appropriately to align each of the extending arms 204 above each of the old prosthetic leaflet of the old prosthetic heart valve.

In some embodiments, the valve delivery system 302 is moved distally (without further deploying the prosthetic heart valve 200) in order to cause engagement of the engaging elements 214 located at the extremities 212 of the extending arms 204 with the external surfaces of old prosthetic leaflets 406, as shown for example in FIG. 4c (508 in FIG. 5).

In some embodiments, the valve delivery system 302 is further moved distally (without further deploying the prosthetic heart valve 200) in order to begin pushing inwards the old prosthetic leaflets 406, (510 in FIG. 5). In some embodiments, the extending arms 204 are actively engaged with old prosthetic leaflets 406, and distal movement of the valve delivery system 302 while keeping the engagement of the extending arms 204 on the old leaflets 406 cause the inward movement of the old leaflets 406, as shown for example in FIG. 4d. It can be seen in FIG. 4d how the extending arms 204 bend due to the forces applied on them by the combination of the movement of the valve delivery system and the engagement of the engaging elements 214 located at the extremities 212 of the extending arms 204.

In some embodiments, the prosthetic heart valve 200 is then deployed from the valve delivery system 302 until the reversibly-compressible ring base 208 of the prosthetic heart valve 200 is positioned below or distally of the old ring base of the old prosthetic heart valve, (512 in FIG. 5). In some embodiments, due to the engagement of the engaging elements 214 located at the extremities 212 of the extending arms 204, the extending arms 204 are bent into a proximal position (the arms extend from the reversibly-compressible ring base 208 in a proximal direction in relation to the orientational positioning of the parts of the device as defined herein), while holding down the inward folded old leaflets of the old prosthetic heart valve. In some embodiments, a potential advantage of positioning the reversibly-compressible ring base 208 of the prosthetic heart valve 200 below or distally of the old ring base of the old prosthetic heart valve is that it potentially avoids generating a high post-procedure transvalvular gradients, therefore providing a wide, larger effective valvular orifice area.

In some embodiments, deployment of the prosthetic heart valve 200 (the change from a crimped configuration to an un-crimped or deployed configuration) causes the body of the prosthetic heart valve 200 to attach to the old prosthetic leaflets of the old heart valve due to the self-expanding nature of the prosthetic heart valve 200, while optionally, engaging the plurality of engaging elements 216 located on the body 202 on one or more of the old prosthetic leaflets of the old heart valve, the body of the old heart valve and the valve posts of the old heart valve, which help anchoring the body of the prosthetic heart valve 200 to the old prosthetic leaflets and therefore to the old prosthetic heart valve (better seen in FIG. 4g).

In some embodiments, a potential advantage of folding inwards the old prosthetic leaflets and then expanding the body of the prosthetic heart valve 200 in order to attach it on those old prosthetic leaflets is that it potentially allows to use the old prosthetic leaflets as a sealing mechanism for the new prosthetic heart valve 200, while also keeping them folded downwards.

In some embodiments, as can be seen in FIGS. 4b-4g, another potential advantage of the valve-in-valve procedure and the prosthetic heart valve 200, is that it potentially allows to replace a failing old prosthetic heart valve with a new prosthetic heart valve without causing coronary occlusion.

In some embodiments, the valve delivery system 302 is retracted leaving the prosthetic heart valve 200 within the old prosthetic heart valve, thereby concluding the valve-in-valve procedure, as shown for example in FIG. 4g (514 in FIG. 5).

### Summary of Exemplary Key Features

In some embodiments, an exemplary feature is related to the nitinol stent frame/body, which comprises one or more limbs that will open during an initial phase of valve deployment. In some embodiments, during this initial phase—the limbs are partially deployed while the new prosthetic heart valve is still within the valve delivery system. In some embodiments, the valve delivery system is optionally rotated appropriately to align each of the extending arms above each of the old prosthetic leaflet of the old prosthetic heart valve. In some embodiments, once aligned with old prosthetic leaflet of the old prosthetic heart valve the extending arms will be lowered into the old prosthetic leaflets. In some embodiments, next, the deployment of the new prosthetic heart valve will begin by exposing the device. In some embodiments, during this process the extending arms push the old prosthetic leaflets downward towards the ventricle. In some embodiments, as the new prosthetic heart valve is being continuously deployed the extending arms will further push down the old prosthetic leaflets of the old prosthetic heart valve until they are at the level of the ring of the old prosthetic heart valve. In some embodiments, by this process, the old prosthetic leaflets will move away and will not obstruct future access to the coronary arteries, furthermore, the old prosthetic leaflets will serve as additional sealing component for the new prosthetic heart valve preventing paravalvular leak.

In some embodiments, another exemplary feature is related to the position in which the new prosthetic heart valve is deployed. In some embodiments, a potential disadvantage of positioning a new prosthetic heart valve within a frame of an old prosthetic heart valve is that it limits the expansion of the new prosthetic heart valve thus limiting the achievable orifice area. In some embodiments, in order to achieve the largest possible effective orifice area of the new prosthetic heart valve is to position the ring of the new prosthetic heart valve below the ring of the old prosthetic heart valve (Sub-annular position). In some embodiments, by this principle, the new prosthetic heart valve will not be constrained by the old prosthetic heart valve. In some embodiments, additionally, according to this principle, the valve mechanism of the new prosthetic heart valve will be located at the bottom of the prosthetic heart valve frame/body, which additionally, is covered by a sealing due to the old prosthetic leaflets that prevent paravalvular leak between the new prosthetic heart valve and the old prosthetic heart valve (in addition to the optional skirt as disclosed above). In some embodiments, the upper part of the new prosthetic heart valve is configured to actuate a strong radial force that serves as an anchoring mechanism within the ring of the old prosthetic heart valve. In some embodiments, the new prosthetic heart valve comprises a strong anchoring mechanism, and the valve itself is below the old prosthetic heart valve and not constrained by its ring.

### Exemplary Add-On In-Folding Leaflets Device

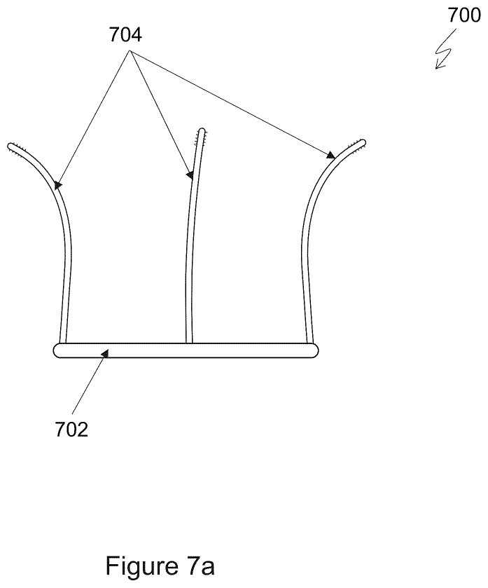

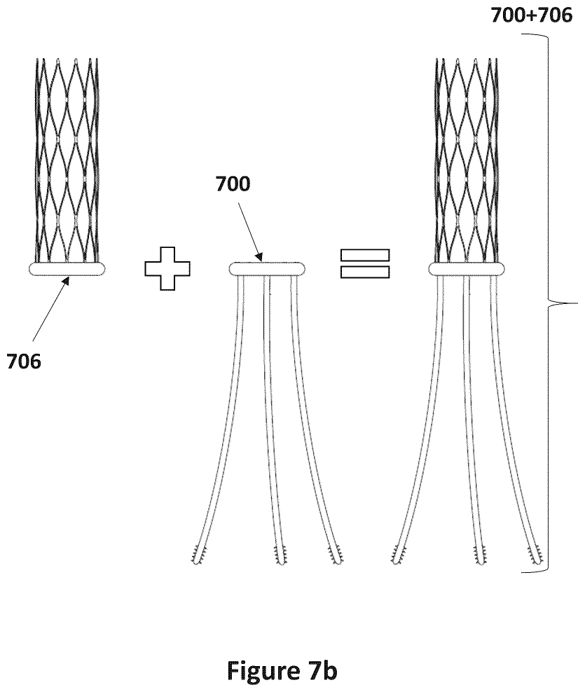

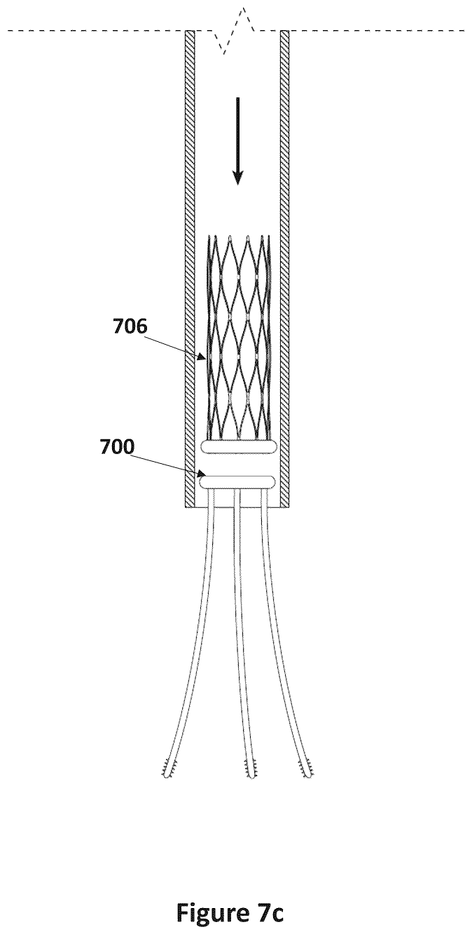

Referring now to FIGS. 7a-c, showing a schematic representation of an exemplary add-on in-folding leaflets device, according to some embodiments of the invention. In some embodiments, when having already a prosthetic heart valve that is wanted to be used, an add-on in-folding leaflets device 700 is used to neutralize the leaflets already found in the site of implantation. In some embodiments, the add-on in-folding leaflets device 700 comprises a ring 702 and one or more extending arms 704, as shown for example in FIG. 7a. In some embodiments, the ring 702 and the one or more extending arms 704 are the same as disclosed above. In some embodiments, the add-on in-folding leaflets device 700 is configured to be mounted and/or coupled on/to an existing prosthetic heart valve 706, which does not have the necessary mechanisms to cause an in-folding of the leaflets, as schematically shown for example in FIG. 7b. In some embodiments, the add-on in-folding leaflets device 700 is used in concomitance with an existing prosthetic heart valve but the two are not coupled to each other. In some embodiments, in this case, the add-on in-folding leaflets device 700 is deployed first, and then the existing prosthetic heart valve is deployed, as schematically shown for example in FIG. 7c. In some embodiments, the methods of deployment are similar to those disclosed above.

As used herein with reference to quantity or value, the term “about” means “within ±20% of”.

The terms “comprises”, “comprising”, “includes”, “including”, “has”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular forms “a”, “an” and “the” include plural references unless the context clearly dictates otherwise. For example, the term “a compound” or “at least one compound” may include a plurality of compounds, including mixtures thereof.

Throughout this application, embodiments of this invention may be presented with reference to a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as “from 1 to 6” should be considered to have specifically disclosed subranges such as “from 1 to 3”, “from 1 to 4”, “from 1 to 5”, “from 2 to 4”, “from 2 to 6”, “from 3 to 6”, etc.; as well as individual numbers within that range, for example, 1, 2, 3, 4, 5, and 6. This applies regardless of the breadth of the range.

Whenever a numerical range is indicated herein (for example “10-15”, “10 to 15”, or any pair of numbers linked by these another such range indication), it is meant to include any number (fractional or integral) within the indicated range limits, including the range limits, unless the context clearly dictates otherwise. The phrases “range/ranging/ranges between” a first indicate number and a second indicate number and “range/ranging/ranges from” a first indicate number “to”, “up to”, “until” or “through” (or another such range-indicating term) a second indicate number are used herein interchangeably and are meant to include the first and second indicated numbers and all the fractional and integral numbers therebetween.

Unless otherwise indicated, numbers used herein and any number ranges based thereon are approximations within the accuracy of reasonable measurement and rounding errors as understood by persons skilled in the art.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

1. (canceled)

2-48. (canceled)

49. A method of implanting a prosthetic heart valve, the prosthetic heart valve having a body, within an existing heart valve having leaflets, the method comprising:
a. outwardly extending at least one extending structure from said body, while the heart valve is at least partially constrained within a valve delivery system;
b. displacing at least one leaflet of said existing heart valve by said at least one extending structure, by advancing said prosthetic heart valve in a ventricular direction;
c. positioning the prosthetic heart valve at an implantation location after and/or during said displacing; and
d. releasing said heart valve from said delivery system and anchoring the heart valve in the implantation location.

50. The method according to claim 49, wherein said advancing comprises advancing said body, constrained within said valve delivery system.

51. The method according to claim 49, wherein said extending comprises actuating said extending structures while said valve is still contained within said valve delivery system.

52. The method according to claim 49, wherein said displacing comprises pushing said leaflets downward in a ventricular direction.

53. The method according to claim 52, wherein said pushing comprises in-folding of the leaflets.

54. The method according to claim 49, wherein said releasing said prosthetic heart valve from said valve delivery system is after said displacing.

55. The method according to claim 54, wherein said releasing initiates the expansion of said heart valve.

56. The method according to claim 55, wherein said expansion results in the anchoring of said prosthetic heart valve in place.

57. The method according to claim 56, wherein said anchoring comprises anchoring in a sub-annular position with regard to said existing heart valve.

58. The method according to claim 49, wherein said releasing further comprises expanding said prosthetic heart valve such that its body engages the in-folded prosthetic leaflets of said existing heart valve, wherein said in-folded prosthetic leaflets are retained in a downward position and form a sealing interface with said prosthetic heart valve.

59. The method according to claim 49, wherein said at least one extending structure is attached to said body.

60. A transcatheter aortic heart valve for implantation into an existing bioprosthetic heart valve having leaflets, the transcatheter aortic heart valve comprising:
a. a frame body comprising a plurality of extending structures;
b. each of the extending structures is connected at its proximal end to a single axial location on said frame body and is constructed to be deployed during an initial phase of deployment while the transcatheter aortic heart valve is still within a valve delivery system;
wherein said extending structures are arranged to align with the leaflets of the existing prosthetic heart valve; and
wherein said extending structures are shaped and positioned to push said leaflets during distal advancement of the prosthetic heart valve.

61. The transcatheter aortic heart valve of claim 60, wherein the frame body is constructed of a self-expanding material.

62. The transcatheter aortic heart valve of claim 60, wherein the frame is metallic and wherein said valve leaflets are sutured thereon.

63. The transcatheter aortic heart valve of claim 60, wherein said body is a cylindrical or tubular body.

64. The transcatheter aortic heart valve of claim 60, wherein the frame body is configured for implantation such that a ring of the transcatheter aortic valve is located in a sub-annular position on the ventricular side of the existing bioprosthetic heart valve.

65. The transcatheter aortic heart valve of claim 60, wherein a valve mechanism of the transcatheter aortic heart valve is located in a lower portion of the frame body adjacent the ventricular side of the existing bioprosthetic heart valve.

66. The transcatheter aortic heart valve of claim 60, wherein each of the extending structures comprises an arm having a length of from about 0.25 cm to about 4 cm.

67. The transcatheter aortic heart valve of claim 60, wherein the extending structures are part of an add-on structure configured to be coupled to a prosthetic heart valve.

68. The transcatheter aortic heart valve of claim 60, wherein said extending structures are flexible arms.

69. The transcatheter aortic heart valve of claim 60, further comprising a sealing skirt.

70. A kit comprising the valve delivery system and the transcatheter aortic heart valve of claim 60, wherein the valve is at least partially contained within the delivery system.
